(19) United States
(12) Patent Application Publication
(10) Pub. No.: US 2010/0010350 A1
(43) Pub. Date: Jan. 14, 2010

(54) ULTRASONIC DIAGNOSTIC APPARATUS AND ULTRASONIC IMAGE ACQUISITION METHOD

(76) Inventors: Tatsuro Baba, Otawara-shi (JP); Hironobu Hongou, Otawara-shi (JP); Takuya Sasaki, Nasu-gun (JP); Shuichi Kawasaki, Nasushiobara-shi (JP); Yuichi Muranaka, Otawara-shi (JP); Masashi Akimoto, Nasushiobara-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/501,736

(22) Filed: Jul. 13, 2009

(30) Foreign Application Priority Data
Jul. 14, 2008 (JP) 2008-183137
Jul. 1, 2009 (JP) 2009-157067

(51) Int. Cl. A61B 8/14

(52) U.S. Cl. 600/443

(57) ABSTRACT

Real-time display of a three-dimensional ultrasonic image in which there is little time delay, power required for transmission of an ultrasonic wave is reduced, and heterogeneousness of received beams is almost solved is performed by executing multi-beam reception while performing frequency demodulation of a reflected wave obtained as a result of transmission of a frequency-modulated continuous ultrasonic wave to the subject body. As a demodulation method, for example, a multi-phase demodulation method of obtaining the distance resolution in multi-beam reception by performing (multi-phase) demodulation while changing the phases of transmission modulation and reception modulation for every range in the range direction (beam direction).

### CROSS-REFERENCE TO RELATED APPLICATIONS

This application is based upon and claims the benefit of priority from prior Japanese Patent Applications No. 2008-183137, filed Jul. 14, 2008; and No. 2009-157067, filed Jul. 1, 2009, the entire contents of both of which are incorporated herein by reference.

### BACKGROUND OF THE INVENTION

1. Field of the Invention

The present invention relates to an ultrasonic diagnostic apparatus and in particular, to an apparatus which executes continuous wave Doppler (CWD) using a continuous wave (CW) as an ultrasonic wave. Specifically, the present invention relates to an ultrasonic diagnostic apparatus and an ultrasonic image acquisition method capable of observing movement information of blood flow, which flows through a specific region in the distance direction, by giving the resolving power in the distance direction while making maximum use of the merit that there is no reflection due to the continuous wave Doppler.

2. Description of the Related Art

The ultrasonic diagnosis allows pulsation of the heart or movement of an embryo to be displayed in real time by a simple operation of bringing an ultrasonic probe into contact with a body surface. In addition, since the ultrasonic diagnosis is very safe, the test may be performed repeatedly. In addition, the system size is small compared with other diagnostic apparatuses, such as an X ray, a CT, and an MRI, and a test can be easily performed at the bedside. For this reason, it can be said that the ultrasonic diagnosis is an easy diagnostic method. An ultrasonic diagnostic apparatus used in the ultrasonic diagnosis changes in various ways with the type of function that the ultrasonic diagnostic apparatus has. As a small ultrasonic diagnostic apparatus, an ultrasonic diagnostic apparatus that can be carried with one hand is being developed. In addition, since the ultrasonic diagnosis does not cause radioactive exposure unlike the X ray, the ultrasonic diagnosis may also be used in an obstetric treatment, a remote medical treatment, and the like.

For example such an ultrasonic diagnostic apparatus may be used to scan a three-dimensional region of the heart to image in three-dimensions in real time. When performing such imaging, there are restrictions in the sound speed in order to obtain volume data by performing ultrasonic scan in a three-dimensional region in real time. Accordingly, there are trade-offs between the volume rate and the depth of field/azimuth resolution (the number of beams of azimuth/elevation). For this reason, there is a problem that sufficient volume rate and azimuth resolution are not obtained, compared with two-dimensional imaging in real time.

In order to solve such a problem, for example, JP-A-2007-215630 discloses a technique of generating a four-dimensional image corresponding to the entire scan range by acquiring data of one cardiac beat in each of different scan ranges by changing the scan start position in a three-dimensional scan range on the basis of an electrocardiographic signal using a two-dimensional array probe, in which ultrasonic vibrators are arrayed in a two-dimensional matrix, and then combining data of the same time phase later. In addition, there is a technique of improving the volume rate by obtaining received beams corresponding to several times the transmitted beams using parallel and simultaneous reception or a technique in which these techniques are combined.

However, there are following problems in the techniques for obtaining the sufficient volume rate and azimuth resolution in real-time display of a three-dimensional image.

First, in the case of using an electrocardiographic signal, a time of a plurality of cardiac beats is required in order to collect volume data. Moreover, in practice, full volume data is generated by connecting a plurality of subvolumes collected corresponding to different cardiac beats. For this reason, since time delay occurs, real-time display is not realized properly.

In addition, in the case of using parallel and simultaneous reception, the volume rate is increased as the number of parallel stages is increased. However, it is necessary to increase the transmission energy in order to cover a wide range of the receiving area. For this reason, a problem of power/generation of heat occurs, and received beams become heterogeneous due to influences of the sound field distribution of transmitted beams. As a result, the quality of an image may be degraded.

### BRIEF SUMMARY OF THE INVENTION

In view of the above, it is an object of the present invention to provide an ultrasonic diagnostic apparatus and an ultrasonic image acquisition method capable of solving heterogeneousness of received beams by reducing time delay and power required for transmission of an ultrasonic wave, compared with those in the related art, in real-time display of a three-dimensional ultrasonic image.

According to an aspect of the present invention, there is provided an ultrasonic diagnostic apparatus including: a transmission unit that transmits a frequency-modulated continuous ultrasonic wave to a predetermined region of a subject body; a reception unit that performs multi-beam reception of a continuous reflected wave generated on the basis of the continuous ultrasonic wave from the predetermined region and outputs a first signal; a signal processing unit that performs frequency demodulation of the first signal and generates a second echo signal separated for every position within the predetermined region; a data generating unit that generates first volume data using the second signal; and an image generating unit that generates an ultrasonic image using the first volume data.

According to another aspect of the present invention, there is provided an ultrasonic diagnostic apparatus including: a transmission unit that transmits a frequency-modulated continuous ultrasonic wave to a predetermined region of a subject body; a reception unit that performs multi-beam reception of a continuous reflected wave generated on the basis of the continuous ultrasonic wave from the predetermined region and outputs a first signal; a signal processing unit that performs frequency demodulation of the first signal for every range in a multi-phase demodulation method and generates a second echo signal separated for every position within the predetermined region; a data generating unit that generates first volume data using the second signal; and an image generating unit that generates an ultrasonic image using the first volume data.

According to yet another aspect of the present invention, there is provided an ultrasonic diagnostic apparatus including: a transmission unit that transmits a frequency-modulated continuous ultrasonic wave to a predetermined region of a subject body; a reception unit that performs multi-beam reception of a continuous reflected wave generated on the basis of the continuous ultrasonic wave from the predetermined region using a first reception delay time regarding a reference beam and a plurality of second delay times determined for every beam as correction delay times for the first reception delay time and that outputs a first signal; a signal processing unit that performs frequency demodulation of the first signal and generates a second echo signal separated for every position within the predetermined region; a data generating unit that generates first volume data using the second signal; and an image generating unit that generates an ultrasonic image using the first volume data.

According to yet another aspect of the present invention, there is provided an ultrasonic diagnostic apparatus including: a transmission unit that transmits a frequency-modulated continuous ultrasonic wave to a predetermined region of a subject body; a reception unit that performs multi-beam reception of a continuous reflected wave generated on the basis of the continuous ultrasonic wave from the predetermined region and outputs a first signal; a signal processing unit that sequentially extracts the first signals, of which frequency bands are different, by performing filtering processing in a time-shared way using a variable band pass filter after performing frequency demodulation of the first signal and generates a second echo signal separated for every position within the predetermined region by executing discrete Fourier transform of the extracted first signals, of which frequency bands are different, in a time-shared way; a data generating unit that generates first volume data using the second signal; and an image generating unit that generates an ultrasonic image using the first volume data.

According to yet another aspect of the present invention, there is provided an ultrasonic image acquisition method including: transmitting a frequency-modulated continuous ultrasonic wave to a predetermined region of a subject body; performing multi-beam reception of a continuous reflected wave generated on the basis of the continuous ultrasonic wave from the predetermined region and outputting a first signal; performing frequency demodulation of the first signal and generating a second echo signal separated for every position within the predetermined region; generating first volume data using the second signal; and generating an ultrasonic image using the first volume data.

### BRIEF DESCRIPTION OF THE SEVERAL VIEWS OF THE DRAWINGS

FIG. 1 is a block diagram illustrating the configuration of an ultrasonic diagnostic apparatus according to the present embodiment;

FIG. 2 is a view for explaining the configuration of an ultrasonic probe provided in the ultrasonic diagnostic apparatus;

FIG. 3 is a view for explaining the configuration of a transmission and reception unit;

FIG. 4 is a flow chart illustrating the flow of process (real-time display process of a three-dimensional image) based on the function of real-time display of a three-dimensional image;

FIG. 5 is a view illustrating the concept of transmission using a frequency modulation method and multi-beam reception for every range using a frequency demodulation method;

FIG. 6 is a view for explaining processing for detecting a fundamental wave component from a beam for every range received using the frequency demodulation method;

FIG. 7 is a view for explaining processing for detecting a fundamental wave component from a beam for every range received using the frequency demodulation method;

FIG. 8 is a view illustrating the hardware configuration regarding reception of a transmission and reception unit of an ultrasonic diagnostic apparatus in the first modification;

FIG. 9 is a view illustrating the hardware configuration regarding reception of the transmission and reception unit of the ultrasonic diagnostic apparatus;

FIG. 10 is a view for explaining frequency demodulation processing in the ultrasonic diagnostic apparatus in the first modification;

FIG. 11 is a view illustrating the hardware configuration regarding reception of a transmission and reception unit of an ultrasonic diagnostic apparatus in the second modification;

FIG. 12 is a view for explaining frequency demodulation processing in the ultrasonic diagnostic apparatus in the second modification;

FIG. 13 is a view illustrating the analog configuration of a DFT in the second modification;

FIG. 14A is a view for explaining a delay method of the ultrasonic diagnostic apparatus in this modification, and FIG. 14B is a view illustrating a delay method in known beam forming; and

FIG. 15 is a view for explaining a phase delay method using a matrix switch.

### DETAILED DESCRIPTION OF THE INVENTION

Hereinafter, embodiments of the present invention will be described with reference to the accompanying drawings. Moreover, in the following description, components having approximately the same function and configuration are denoted by the same reference numeral, and a repeated explanation thereof will only be made when necessary.

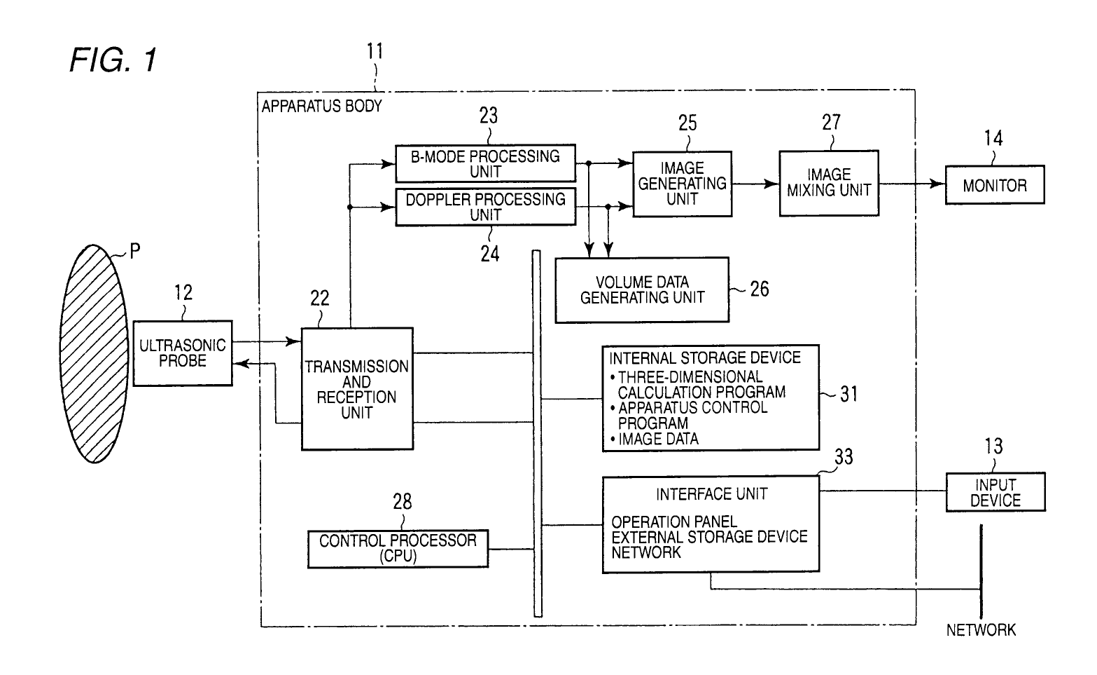

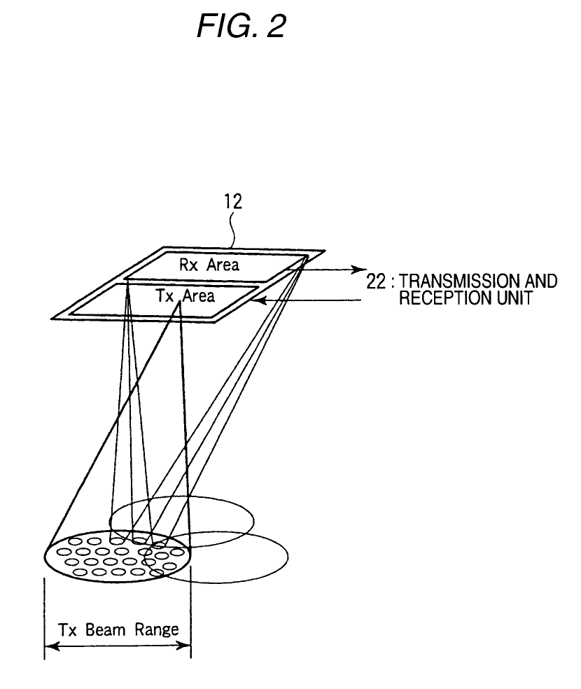

FIG. 1 is a block diagram illustrating the configuration of an ultrasonic diagnostic apparatus 11 according to the present embodiment. As shown in FIG. 1, the ultrasonic diagnostic apparatus 11 includes an ultrasonic probe 12, an input device 13, a monitor 14, an ultrasonic wave transmission and reception unit 22, a B-mode processing unit 23, a Doppler processing unit 24, an image generating unit 25, a volume data generating unit 26, an image mixing unit 27, a control processor (CPU) 28, an internal storage unit 31, and an interface unit 33. Hereinafter, a function of each constituent component of the ultrasonic diagnostic apparatus 11 will be described.

The ultrasonic probe 12 generates an ultrasonic wave on the basis of a driving signal from the ultrasonic wave transmission and reception unit 22 and has a plurality of piezoelectric vibrators that convert a reflected wave from a subject body into an electric signal, a matching layer provided in the piezoelectric vibrators, a backing material for preventing propagation of an ultrasonic wave rearward from the piezoelectric vibrators, and the like. When an ultrasonic wave is transmitted from the ultrasonic probe 12 to a subject body P, the transmitted ultrasonic wave is sequentially reflected on a discontinuous surface of acoustic impedances of body tissues and is then received as an echo signal by the ultrasonic probe 12. The amplitude of the echo signal depends on the difference of the acoustic impedances on the discontinuous surfaces on which the ultrasonic wave is reflected. In addition, an echo when transmitted ultrasonic pulses are reflected by a moving blood flow, on surface of a heart wall, and the like is frequency-shifted depending on a speed component of the moving body in the ultrasonic wave transmission direction by the Doppler effect.

In addition, the ultrasonic probe 12 provided in the ultrasonic diagnostic apparatus is a two-dimensional-array probe in which a plurality of ultrasonic vibrators are arrayed in a two-dimensional matrix. Moreover, the ultrasonic probe 12 has a transmission area (Tx Area) and a receiving area (Rx Area), each of which is configured to include a plurality of ultrasonic vibrators, in order to execute transmission and reception of a continuous ultrasonic wave in parallel. A frequency-modulated continuous ultrasonic wave is transmitted in the transmission area, and multi-beam reception is executed while performing frequency demodulation in the receiving area. This will be described in detail later.

The input device 13 is connected to an apparatus body 11 and has various switches, buttons, a track ball, a mouse, a keyboard, and the like used to perform various instructions from an operator, an instruction for setting a condition or a region of interest (ROI), an instruction for setting various conditions of image quality, and the like on the apparatus body 11. For example, when the operator operates a stop button or a ‘FREEZE’ button of the input device 13, transmission and reception of an ultrasonic wave are stopped and the ultrasonic diagnostic apparatus is temporarily stopped.

The monitor 14 displays morphological information (a normal B-mode image) in the body, blood flow information (including an average speed image, a dispersion image, a power image), and the like in predetermined forms on the basis of a video signal from the scan converter 25.

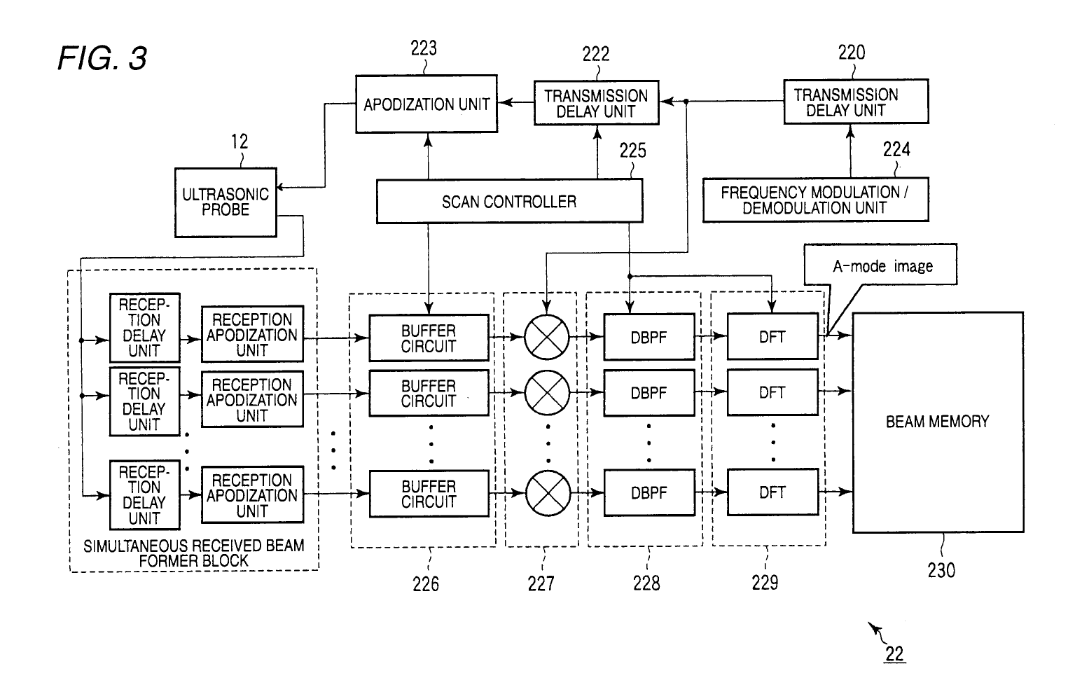

The ultrasonic wave transmission and reception unit 22 has a signal generator 220, a transmission mixer 221, a transmission delay unit 222, an apodization unit 223, a frequency modulation/demodulation unit 224, a receiving buffer unit 226, a receiving mixer 227, a DBPF 228, a discrete Fourier transform unit 229, and a beam memory 230, as shown in FIG. 3. The ultrasonic wave transmission and reception unit 22 not only performs transmission and reception of an ultrasonic wave in a normal B mode, a pulse Doppler mode, or the like, but also performs multi-beam reception while performing frequency demodulation of a reflected wave obtained as a result of transmission of a frequency-modulated continuous ultrasonic wave in the case of executing real-time display of a three-dimensional image.

The B-mode processing unit 23 receives an echo signal from the transmission and reception unit 22, performs logarithmic amplification, envelope detection processing, and the like, and generates data in which the signal strength is expressed as a brightness level. This data is transmitted to the scan converter 25 and is displayed on the monitor 14 as a B-mode image which expresses the strength of a reflected wave by the brightness.

The Doppler processing unit 24 performs a frequency analysis of speed information from the echo signal received from the transmission and reception unit 22, extracts a blood flow or a tissue and a contrast echo component caused by the Doppler effect, and calculates blood flow information, such as an average speed, dispersion, and power, for multiple points. In particular, the Doppler processing unit 24 reads multi-phase demodulated data sequentially from the transmission and reception unit 22, calculates the spectrum obtained in each range, and calculates data of a CW spectrum image using the spectrum.

The image generating unit 25 generates an ultrasonic image using the data received from the B-mode processing unit 23, the Doppler processing unit 24, and the volume data generating unit 26.

The volume data generating unit 26 generates volume data corresponding to each time phase by connecting subvolume data using a function of real-time display of a three-dimensional image, which will be described later.

The image mixing unit 27 mixes an image received from the image generating unit 25 with alphabetic information, scales, or the like of various parameters and outputs the mixing result as a video signal to the monitor 14.

The control processor 28 has a function as an information processing device (computer) and controls an operation of the main body of the ultrasonic diagnostic apparatus. The control processor 28 reads an exclusive program for realizing a function of real-time display of a three-dimensional image, which will be described later, and a control program for executing a predetermined scan sequence from the internal storage unit 31, loads the read programs onto its own memory, and executes an operation, control, and the like regarding various kinds of processing.

The internal storage unit 31 stores a predetermined scan sequence for collecting a plurality of volume data by the setting of different angles of view, an exclusive program for realizing a function of real-time display of a three-dimensional image which will be described later, a control program for executing image generation and display processing, diagnostic information (for example, a patient ID or doctor's opinion), a diagnostic protocol, transmission and reception conditions, a body mark creating program, and other data groups. In addition, the internal storage unit 31 may be used to store an image in the image memory 26 as necessary. Data stored in the internal storage unit 31 may be transmitted to an external peripheral device through the interface unit 33.

The interface unit 33 is an interface related to the input device 13, a network, and a new external storage device (not shown). Data, an analysis result, or the like of an ultrasonic image and the like obtained by the apparatus may be transmitted to another apparatus through the network by the interface unit 33.

(Function of Real-Time Display of a Three-Dimensional Image)

Next, a function of real-time display of a three-dimensional image that the ultrasonic diagnostic apparatus 11 has will be described. This function is to perform real-time display of a three-dimensional ultrasonic image in which there is little time delay, power required for transmission of an ultrasonic wave is reduced, and heterogeneousness of received beams is almost solved by performing multi-beam reception while performing frequency demodulation of a reflected wave obtained as a result of transmission of a frequency-modulated continuous ultrasonic wave to the subject body. Moreover, in the present embodiment, a multi-phase demodulation method is adopted as a demodulation method in order to make a specific explanation. This is to obtain the distance resolution in multi-beam reception by performing (multi-phase) demodulation while changing the phases of transmission modulation and reception modulation for every range in the range direction (beam direction). However, the technical spirit of the present invention is not limited to the multi-phase demodulation method, but any demodulation method may be adopted as long as the substantially same result as in the multi-phase demodulation method can be acquired.

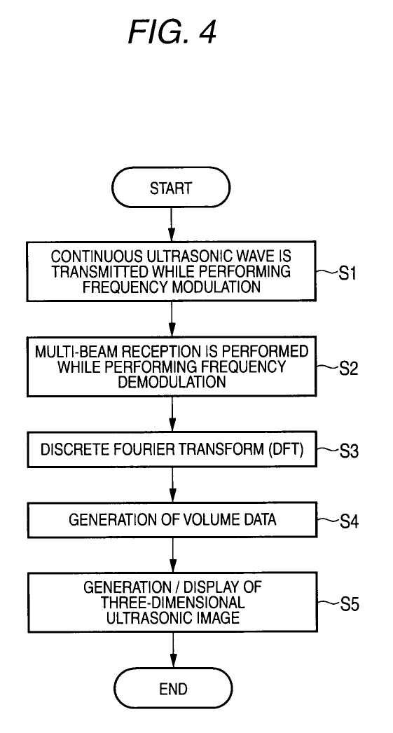

FIG. 4 is a flow chart illustrating the flow of process (real-time display process of a three-dimensional image) based on the function of real-time display of a three-dimensional image. Hereinafter, the real-time display process of a three-dimensional image will be described according to the flow chart.

[Continuous Ultrasonic Wave Transmission Using a Frequency Modulation Method: Step S1]

First, transmission of a continuous ultrasonic wave, the transmission voltage for which is low (power/generation of heat is small), is executed using the frequency modulation method (step S1). That is, the signal generator 220 performs frequency modulation using the frequency modulation/demodulation unit 224. In addition, a transmission delay time is given to the frequency-modulated signal by the transmission delay unit 222 and beam directivity is given to the frequency-modulated signal by the apodization unit 223, and then the frequency-modulated signal is supplied to the ultrasonic probe 12. This supply is executed in a sequential manner. The ultrasonic probe 12 transmits the continuous ultrasonic wave (continuous wave chirp) into the subject body on the basis of the supplied signal.

In addition, in order to realize ultrasonic scan in a wide range, it is preferable that the continuous ultrasonic wave be transmitted in the shape of a fan beam. In addition, in order to prevent the electric power from concentrating on one element, it is preferable that the continuous ultrasonic wave of a fan beam be transmitted while changing the direction.

[Multi-Beam Reception Using a Frequency Demodulation Method: Step S2]

Next, multi-beam reception using a frequency demodulation method is executed (step S2). That is, the ultrasonic probe 12 performs multi-beam reception of a reflected wave from the inside of the subject body based on the transmitted continuous ultrasonic wave for every range direction. The receiving buffer unit 226 performs A/D conversion of each of beams which were received as multiple beams in a buffer circuit provided corresponding to each range and stores it as digital data first. In addition, since the receiving buffer unit 226 functions as a buffer connected with time delay, sample data of multi-phases corresponding to the resolving power in the range direction is mapped as much as the Nfm period of FM modulation by related storage. The receiving mixer 227 and the frequency modulation/demodulation unit 224 execute multi-phase FM demodulation in synchronization with transmission modulation for every range direction. Here, ‘synchronization’ means performing FM demodulation with a chirp wave synchronized with a chirp wave for frequency modulation with respect to a transmitted continuous wave with a fixed time interval therebetween. Moreover, as a measure against saturation of an echo signal from the short distance when performing reception in this step, it is preferable to perform a TGC (time gain control) according to a modulation period frequency.

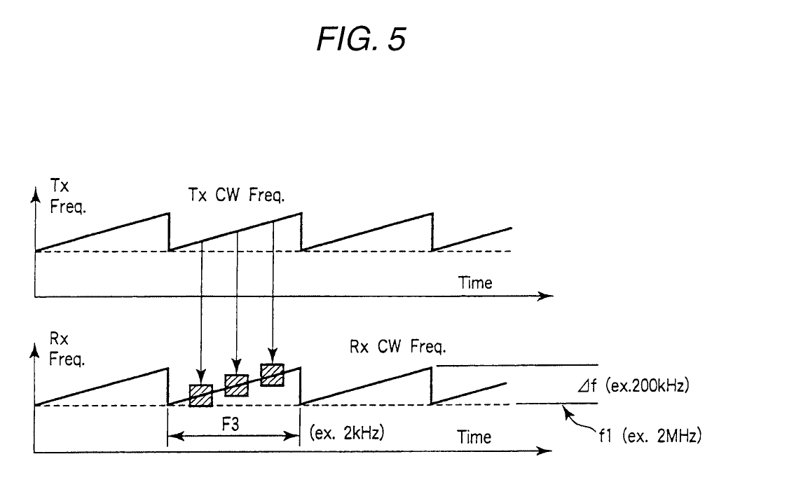

By such a multi-phase FM demodulation method, as shown in FIG. 5, reflected waves based on continuous ultrasonic waves transmitted using a frequency modulation method are received such that the phase of transmission modulation and the phase of reception modulation are changed for every range and the reflected waves are synchronized with a fixed time interval therebetween.

[Discrete Fourier Transform: Step S3]

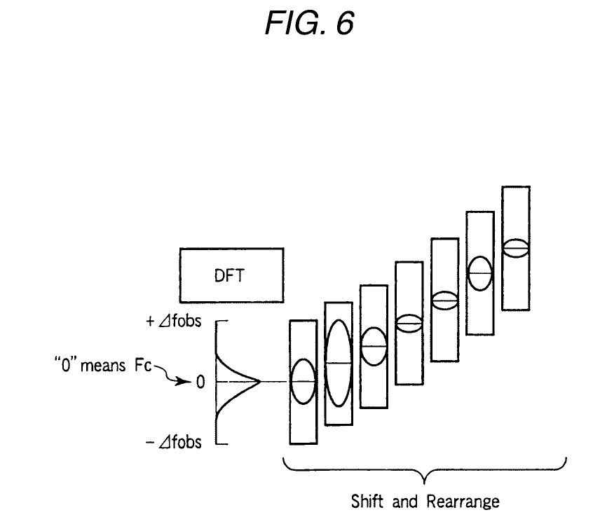

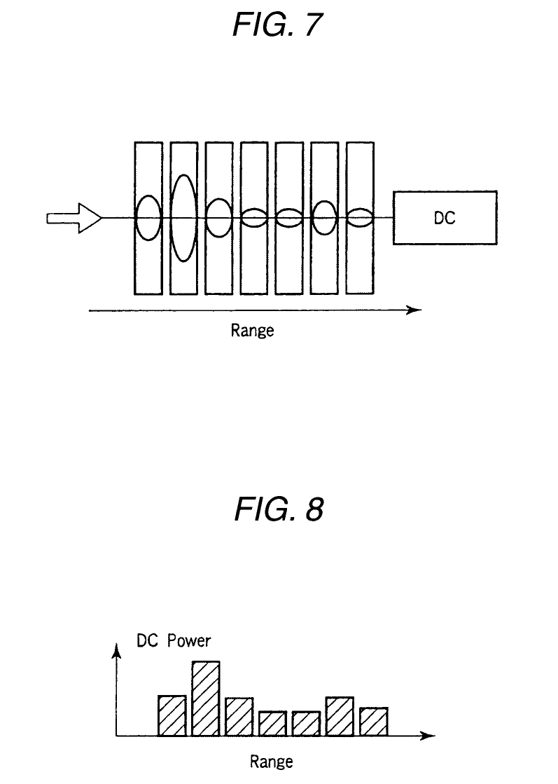

Next, discrete Fourier transform is executed for each of beams received as multiple beams by using a frequency demodulation method, and a fundamental wave for every range is extracted (step S3). That is, the discrete Fourier transform unit 229 executes discrete Fourier transform for each beam in each range filtered by the band filter unit 228 and calculates the spectrum of each beam in each range as shown in FIG. 6 (step S3). The beam memory 230 rearranges the calculated spectrum of fundamental wave components of each beam with a range as a reference as shown in FIG. 7 and then acquires fundamental wave power (that is, A-mode information for every range) for every range as it is shown in FIG. 8. As a result, an echo signal is extracted for every distance.

[Generation of Volume Data: Step S4]

Next, processing of steps S1 to S3 is executed sequentially and continuously, such that subvolume data for a predetermined region is sequentially generated. Moreover, if necessary, received signal gain correction processing for correcting the sensitivity difference within the subvolume and the sensitivity difference between subvolumes caused by interpolation processing and the like is executed. Moreover, if necessary, in order to improve the S/N ratio, a modulated wave may be applied for subvolume data a plural number of times so that the subvolume data is temporally mixed. The volume data generating unit 26 generates volume data by aligning and connecting the subvolumes obtained in this way (step S4).

[Generation/Display of a Three-Dimensional Image: Step S5]

Next, the image generating unit 25 generates a three-dimensional image using the generated volume data (step S5). The generated three-dimensional image is mixed with required information in the image mixing unit 27 and is then displayed on the monitor 14 in a predetermined form (step S5).

(First Modification)

Next, the first modification of the ultrasonic diagnostic apparatus 11 according to the present embodiment will be described. The ultrasonic diagnostic apparatus 11 in the first modification is an example in which the entire hardware size is reduced particularly by integrating circuits related to reception.

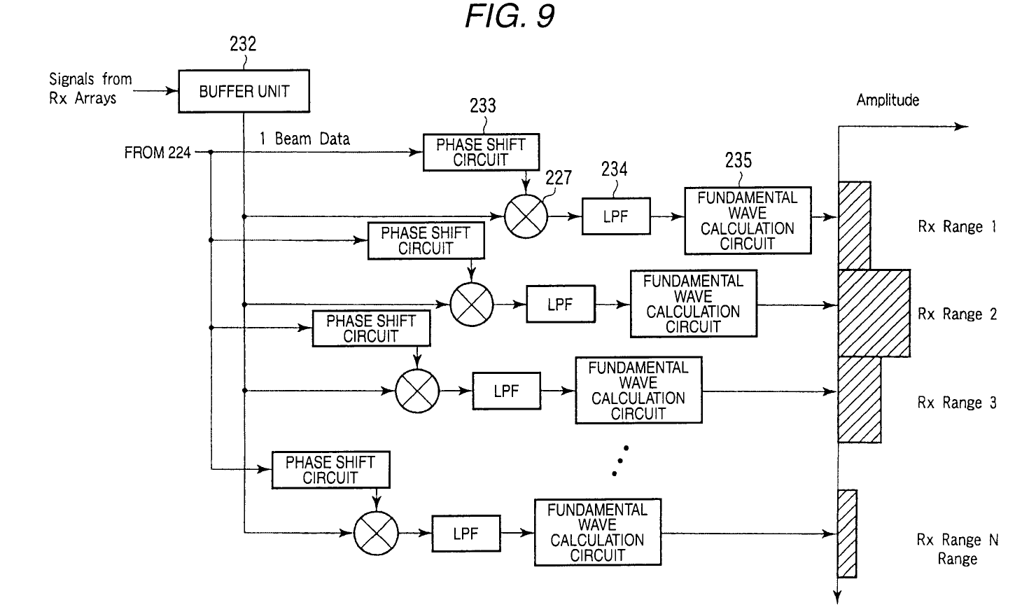

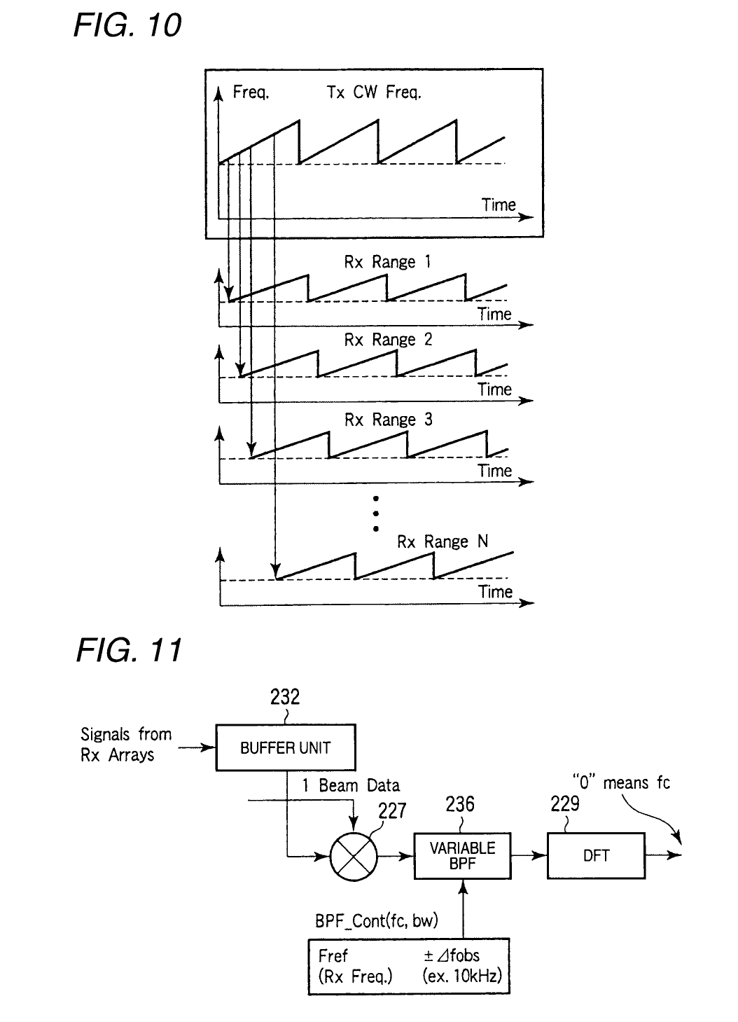

FIG. 9 is a view illustrating the hardware configuration regarding reception of the transmission and reception unit 22 of the ultrasonic diagnostic apparatus 11. As shown in FIG. 9, echo signals received by a plurality of ultrasonic vibrators are A/D converted in the buffer unit 232, are stored as digital data first, and are transmitted to the receiving mixer 227 corresponding to each range. A phase shift circuit 233 controls the operation timing of the receiving mixer 227 on the basis of a control signal from the frequency modulation/demodulation unit 224, and executes multi-phase FM demodulation in synchronization with transmission modulation for every range direction as shown in FIG. 10. A fundamental wave calculation circuit 235 corresponding to each range calculates the amplitude of a fundamental wave for every range using each beam for every range filtered by a LPF (low pass filter) 234.

According to the above configuration, the hardware size can be reduced, and the multi-beam reception can be performed while performing multi-phase demodulation of a reflected wave obtained as a result of transmission of a frequency-modulated continuous ultrasonic wave to the subject body.

(Second Modification)

Next, the second modification of the ultrasonic diagnostic apparatus 11 according to the present embodiment will be described. The ultrasonic diagnostic apparatus 11 in the second modification is an example in which the hardware size related to reception is reduced by narrowing components to be detected to clutter components (that is, by making Doppler components not included).

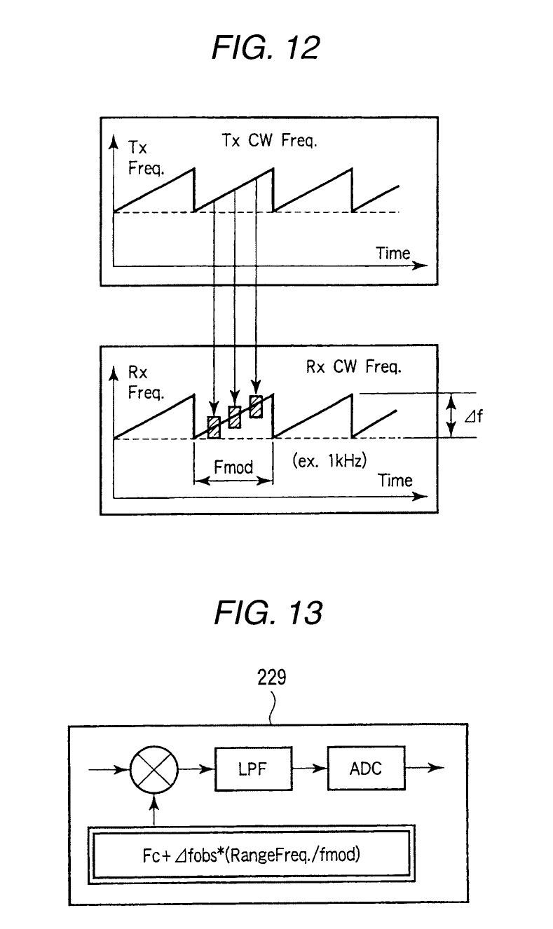

FIG. 11 is a view illustrating the hardware configuration regarding reception of the transmission and reception unit 22 of the ultrasonic diagnostic apparatus 11. In this modification, components to be detected are narrowed to clutter components. Accordingly, by using a variable BPF (band pass filter) 236 and a DFT 229 in a time-shared way as shown in FIG. 11, signal processing for a plurality of ranges can be performed in one system. That is, the variable BPF 236 performs frequency demodulation of a received fundamental frequency Freq. as shown in FIG. 12 while executing filtering for making a signal, which corresponds to a band having a width of Δf from the received fundamental frequency Freq., pass the variable BPF 236 according to the control from frequency modulation/demodulation unit 224. The DFT 229 calculates the amplitude of a clutter component for every range using each beam for every range filtered in the variable BPF 236. In this case, it is preferable to calculate the total power of beams corresponding to each range. In addition, the DFT 229 may be replaced with an analog configuration shown in FIG. 13.

Also by the above configuration, the hardware size can be reduced, and a clutter component can be detected by performing multi-beam reception while performing multi-phase demodulation of a reflected wave obtained as a result of transmission of a frequency-modulated continuous ultrasonic wave to the subject body.

(Third Modification)

Next, the third modification of the ultrasonic diagnostic apparatus 11 according to the present embodiment will be described. In the case of the ultrasonic diagnostic apparatus 11 in the third modification, the hardware size is reduced by configuring a delay method when performing multi-beam reception with delay of a center beam and correction delay of a peripheral beam. That is, it is another method for realizing apodization data or delay data of reception apodization and reception delay unit of a beam former of parallel and simultaneous reception shown in FIG. 3.

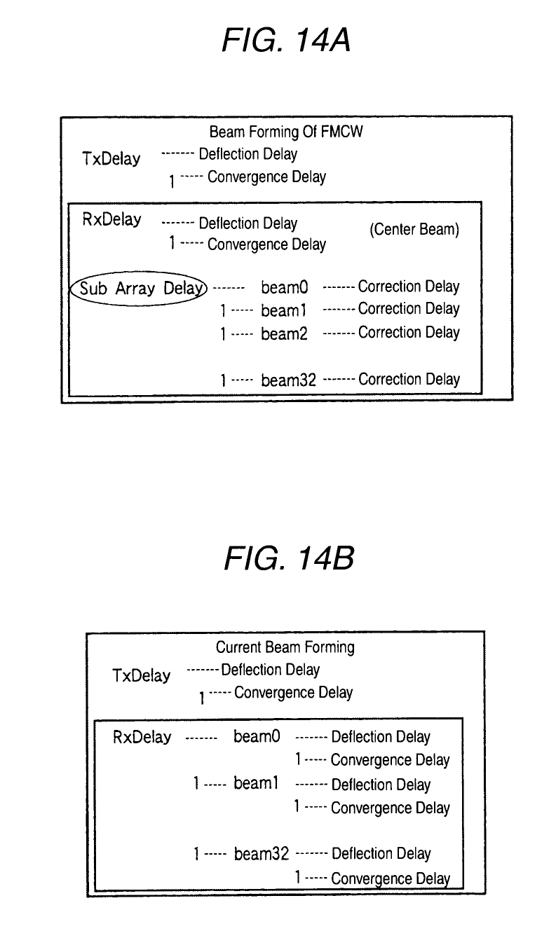

FIG. 14A is a view for explaining a delay method of the ultrasonic diagnostic apparatus 11 in this modification, and FIG. 14B is a view illustrating a delay method in known beam forming. As shown in FIG. 14B, in the known delay method, deflection delay and convergence delay are defined for every beam corresponding to each range independently. On the other hand, in this modification, as shown in FIG. 14A, deflection delay and convergence delay regarding the center beam are defined and deflection delay and convergence delay for every beam corresponding to each range are defined as amounts of deviation therefrom. In addition, deflection delay and convergence delay for every beam corresponding to each range with the center beam as a reference are stored beforehand in the internal storage unit 31 for various conditions, for example.

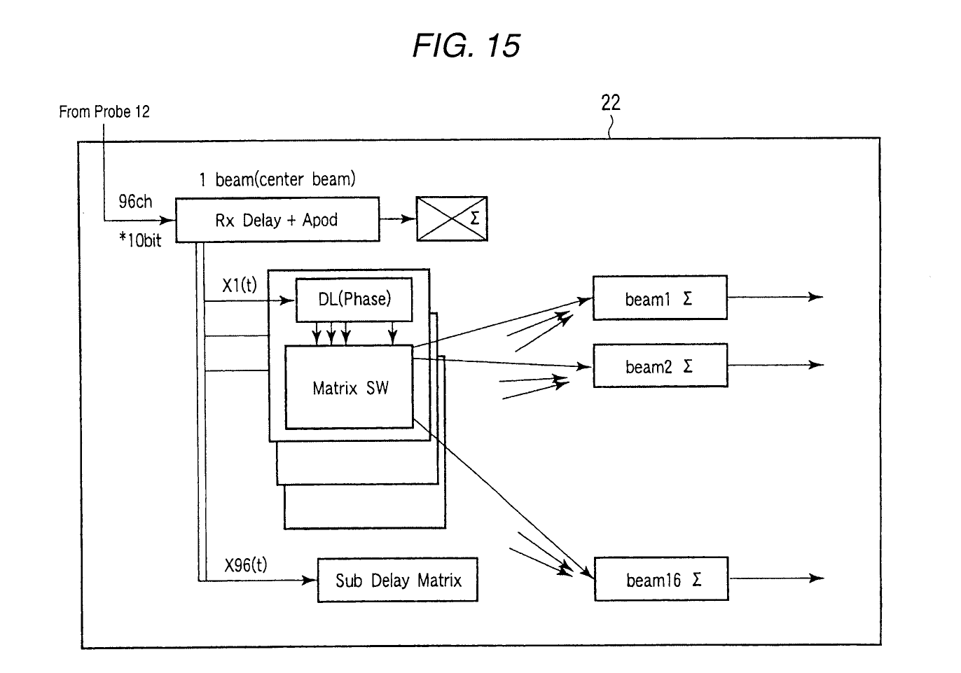

In addition, when performing the above-described beam delay, it is preferable to realize phase delay using a matrix switch (multi-input multi-output switch) shown in FIG. 15. According to such a configuration, the performance can be significantly improved compared with pulse phase delay. In addition, the matrix switch is a multi-input multi-output switch by which delay signals corresponding to delay values can be programmably switching-input to a plurality of beam sum inputs.

(Effects)

According to the ultrasonic diagnostic apparatus of the present embodiment, multi-beam reception is performed while performing frequency demodulation of a reflected wave obtained as a result of transmission of a continuous ultrasonic wave, the transmission voltage for which is low, by using the frequency demodulation method. Accordingly, generation of heat caused by transmission power can be suppressed. In addition, discrete Fourier transform and the like are executed for each of beams received as multiple beams using the frequency demodulation method, an echo signal for every distance corresponding to each range is detected, and a three-dimensional image is displayed in real time using it. Accordingly, since an echo signal having distance resolution can be acquired by scanning an ultrasonic wave in a wide range, a three-dimensional image can be generated and displayed in real time without requiring the time of a plurality of cardiac beats.

In addition, the present invention is not limited to the above-described embodiment and may be embodied in practice by modifying constituent components without departing from the scope and spirit of the present invention. For example, specific modifications include the following examples.

(1) Each of the functions in the present embodiment may be realized by installing a program for executing corresponding processing in a computer, such as a workstation, and then loading the program into the memory. In this case, a program capable of causing a computer to execute a corresponding technique may be distributed in a state where the program is stored in a recording medium, such as a magnetic disk (for example, a floppy (registered trademark) disk or a hard disk), an optical disk (for example, a CD-ROM or a DVD), and a semiconductor memory.

(2) In the above-described embodiment, Doppler information may be acquired and displayed by performing triangular wave modulation, for example, without performing saw-tooth wave modulation shown in FIG. 5 and the like. In addition, the saw-tooth wave may be transmitted and received intermittently according to the required depth of field. In addition, a modulated wave may be made to have a curve to increase the short-distance resolution.

(3) In the above-described embodiment, when calculating A-mode information from a received beam, DC component power may be calculated only by a digital mixer. The hardware size can be further reduced by adopting the above-described configuration. In addition, calculating A-mode information from a received beam may also be realized by using an LPF which extracts only a DC component after an analog mixer.

(4) In the above-described embodiment, the case in which the technical spirit of the present invention was applied to the ultrasonic diagnostic apparatus as a function of real-time display of a three-dimensional image was exemplified. However, the technical spirit of the present invention is not limited to the example of application to the ultrasonic diagnostic apparatus. For example, the technical spirit of the present invention may also be applied to an apparatus which gives the alarm for preventing collision of a moving object (for example, an automobile) using an ultrasonic wave. In this case, not only an ultrasonic image but also a sound or the like which warns the possibility of collision may be output as information supplied to the operator in real time.

In addition, various kinds of inventions may be realized by proper combination of the plurality of constituent components disclosed in the embodiment described above. For example, some constituent components may be eliminated from all components shown in the above embodiment. Moreover, the constituent components in different embodiments may be appropriately combined.

What is claimed is:

1. An ultrasonic diagnostic apparatus comprising:
a transmission unit that transmits a frequency-modulated continuous ultrasonic wave to a predetermined region of a subject body;
a reception unit that performs multi-beam reception of a continuous reflected wave generated on the basis of the continuous ultrasonic wave from the predetermined region and outputs a first signal;
a signal processing unit that performs frequency demodulation of the first signal and generates a second echo signal separated for every position within the predetermined region;
a data generating unit that generates first volume data using the second signal; and
an image generating unit that generates an ultrasonic image using the first volume data.

2. The ultrasonic diagnostic apparatus according to claim 1,
wherein the transmission unit transmits the continuous ultrasonic wave into the predetermined region in the shape of a fan beam.

3. The ultrasonic diagnostic apparatus according to claim 2,
wherein the transmission unit transmits the continuous ultrasonic wave to each of a plurality of subregions, which forms the predetermined region, by switching of transmission direction of the fan beam.

4. The ultrasonic diagnostic apparatus according to claim 1,
wherein the transmission unit transmits the continuous ultrasonic wave to each of a plurality of subregions, which forms the predetermined region, a plural number of times, and
the reception unit outputs the temporally mixed first signal using the continuous reflected wave corresponding to each of the plural number of transmissions.

5. The ultrasonic diagnostic apparatus according to claim 1,
wherein the frequency modulation is saw-tooth modulation.

6. The ultrasonic diagnostic apparatus according to claim 1,
wherein the frequency modulation is triangular wave modulation.

7. The ultrasonic diagnostic apparatus according to claim 1,
wherein the frequency modulation is curve wave modulation.

8. The ultrasonic diagnostic apparatus according to claim 1,
wherein the transmission unit uses saw-tooth modulation intermittently according to depth of field.

9. The ultrasonic diagnostic apparatus according to claim 3,
wherein the reception unit performs multi-beam reception of the continuous reflected wave for each of the subregions and outputs the first signal corresponding to each subregion,
the signal processing unit performs frequency demodulation of the first echo signal corresponding to each subregion and generates the second signal corresponding to each subregion, and
the data generating unit generates the first volume data using the second signal for each of the subregions.

10. The ultrasonic diagnostic apparatus according to claim 9,
wherein the signal processing unit corrects a sensitivity difference between the first echo signals corresponding to the respective subregions.

11. The ultrasonic diagnostic apparatus according to claim 1,
wherein the signal processing unit performs frequency demodulation of the first signal for every range using a multi-phase demodulation method.

12. The ultrasonic diagnostic apparatus according to claim 1,
wherein the signal processing unit executes discrete Fourier transform after performing frequency demodulation of the first signal and generates the second signal including amplitude information using the first signal after the discrete Fourier transform.

13. The ultrasonic diagnostic apparatus according to claim 1,
wherein the signal processing unit extracts a fundamental wave component from the first signal using a digital mixer and generates the second signal including amplitude information on the basis of the extracted fundamental wave component.

14. The ultrasonic diagnostic apparatus according to claim 1,
wherein the signal processing unit extracts a fundamental wave component from the first signal using a low pass filter after processing using an analog mixer and generates the second signal including amplitude information on the basis of the extracted fundamental wave component.

15. The ultrasonic diagnostic apparatus according to claim 1,
wherein the signal processing unit sequentially extracts the first signals, of which frequency bands are different, by performing filtering processing in a time-shared way using a variable band pass filter after performing frequency demodulation of the first signal and generates the second signal by executing discrete Fourier transform of the extracted first signals, of which frequency bands are different, in a time-shared way.

16. The ultrasonic diagnostic apparatus according to claim 1,
wherein the signal processing unit executes a time gain control in synchronization with the frequency demodulation.

17. The ultrasonic diagnostic apparatus according to claim 1,
wherein the reception unit executes the multi-beam reception using a first reception delay time regarding a reference beam of the multiple beams and a plurality of second delay times determined for every beam as correction delay times for the first reception delay time.

18. The ultrasonic diagnostic apparatus according to claim 17,
wherein the reception unit executes multi-beam reception using the first reception delay time and the plurality of second reception delay times by using matrix switching.

19. An ultrasonic diagnostic apparatus comprising:
a transmission unit that transmits a frequency-modulated continuous ultrasonic wave to a predetermined region of a subject body;
a reception unit that performs multi-beam reception of a continuous reflected wave generated on the basis of the continuous ultrasonic wave from the predetermined region and outputs a first signal;
a signal processing unit that performs frequency demodulation of the first signal for every range in a multi-phase demodulation method and generates a second echo signal separated for every position within the predetermined region;
a data generating unit that generates first volume data using the second signal; and
an image generating unit that generates an ultrasonic image using the first volume data.

20. An ultrasonic diagnostic apparatus comprising:
a transmission unit that transmits a frequency-modulated continuous ultrasonic wave to a predetermined region of a subject body;
a reception unit that performs multi-beam reception of a continuous reflected wave generated on the basis of the continuous ultrasonic wave from the predetermined region using a first reception delay time regarding a reference beam and a plurality of second delay times determined for every beam as correction delay times for the first reception delay time and that outputs a first signal;
a signal processing unit that performs frequency demodulation of the first signal and generates a second echo signal separated for every position within the predetermined region;
a data generating unit that generates first volume data using the second signal; and
an image generating unit that generates an ultrasonic image using the first volume data.

21. An ultrasonic diagnostic apparatus comprising:
a transmission unit that transmits a frequency-modulated continuous ultrasonic wave to a predetermined region of a subject body;
a reception unit that performs multi-beam reception of a continuous reflected wave generated on the basis of the continuous ultrasonic wave from the predetermined region and outputs a first signal;
a signal processing unit that sequentially extracts the first signals, of which frequency bands are different, by performing filtering processing in a time-shared way using a variable band pass filter after performing frequency demodulation of the first signal and generates a second echo signal separated for every position within the predetermined region by executing discrete Fourier transform of the extracted first signals, of which frequency bands are different, in a time-shared way;
a data generating unit that generates first volume data using the second signal; and
an image generating unit that generates an ultrasonic image using the first volume data.

22. An ultrasonic image acquisition method comprising:
transmitting a frequency-modulated continuous ultrasonic wave to a predetermined region of a subject body;
performing multi-beam reception of a continuous reflected wave generated on the basis of the continuous ultrasonic wave from the predetermined region and outputting a first signal;
performing frequency demodulation of the first signal and generating a second echo signal separated for every position within the predetermined region;
generating first volume data using the second signal; and
generating an ultrasonic image using the first volume data.
